(19) United States
(12) Patent Application Publication
(10) Pub. No.: US 2024/0092772 A1
(43) Pub. Date: Mar. 21, 2024

(54) CRYSTALLINE FORM OF A PIPERAZINYL-THIAZOLE DERIVATIVE

(71) Applicant: Idorsia Pharmaceuticals Ltd, Allschwil (CH)

(72) Inventor: Dominik FOSSHAG, Allschwil (CH)

(21) Appl. No.: 18/263,318

(22) PCT Filed: Jan. 26, 2022
(86) PCT No.: PCT/EP2022/051786

§ 371 Date: Jul. 27, 2023

(30) Foreign Application Priority Data
Jan. 28, 2021 (WO) PCT/EP2021/051983

(51) Int. Cl. C07D 417/14

(52) U.S. Cl. CPC C07D 417/14; C07B 2200/13

(57) ABSTRACT

The invention relates to a crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, processes for the preparation thereof, pharmaceutical compositions comprising said crystalline form, pharmaceutical compositions prepared from such crystalline forms, and their use as CXCR3 receptor modulators in the treatment of various diseases and disorders related to the CXCR3 receptor and its ligands.

The invention relates to a crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone (hereinafter also referred to as “COMPOUND”), processes for the preparation thereof, pharmaceutical compositions comprising said crystalline form, pharmaceutical compositions prepared from such crystalline forms, and their use as CXCR3 receptor modulators in the treatment of various diseases and disorders related to the CXCR3 receptor and its ligands. In particular, the COMPOUND in crystalline form may be used alone or in pharmaceutical compositions for the prevention/prophylaxis or treatment of diseases and disorders comprising (auto-)immune/inflammatory mediated disorders; pulmonary disorders; cardiovascular disorders; infectious diseases; fibrotic disorders; neurodegenerative disorders; and tumor diseases; and especially of rheumatoid arthritis, multiple sclerosis, Crohn's disease, ulcerative colitis, systemic lupus erythematosus, lupus nephritis, sarcoidosis, systemic sclerosis, psoriasis, psoriatic arthritis, interstitial cystitis, celiac disease, myasthenia gravis, type I diabetes, vitiligo, uveitis, inflammatory myopathies, dry eye disease, thyroiditis including Grave's disease, transplant rejection, acute and/or chronic graft versus host disease, acute lung injury, acute respiratory distress syndrome, asthma, chronic obstructive pulmonary disorder, atherosclerosis, myocarditis, influenza, cerebral malaria, liver cirrhosis, Alzheimer's disease, neurodegeneration, Huntington's chorea, neuromyelitis optica, chronic inflammatory demyelinating polyneuropathy, Guillain-Barré syndrome, brain tumor, colon cancer, breast cancer, and/or metastatic spread of cancer.

### BACKGROUND OF THE INVENTION

Chemokine receptors are a group of G-protein coupled receptors (GPCRs) that bind peptidic chemokine ligands with high affinity. The predominant function of chemokine receptors is to guide leukocyte trafficking to lymphoid organs and tissues under resting conditions as well as during inflammation, but a role for certain chemokine receptors on non-hematopoietic cells and their progenitors has also been recognized.

The chemokine receptor CXCR3 is a G-protein coupled receptor binding to the inflammatory chemokines CXCL9 (initially called MIG, monokine induced by interferon-γ [INF-γ]), CXCL10 (IP-10, INF-γ-inducible protein 10), and CXCL11 (I-TAC, INF-γ-inducible T cell α chemo-attractant). CXCR3 is mainly expressed on activated T helper type 1 (Th1) lymphocytes, but is also present on natural killer cells, macrophages, dendritic cells and a subset of B lymphocytes. The three CXCR3 ligands are expressed mainly under inflammatory conditions, expression in healthy tissue is very low. Cells that can express CXCR3 ligands, for instance after exposure to inflammatory cytokines such as interferon-γ or TNF-α, include diverse stromal cells such as endothelial cells, fibroblasts, epithelial cells, keratinocytes but also includes hematopoietic cells such as macrophages and monocytes. The interaction of CXCR3 and its ligands (henceforth referred to as the CXCR3 axis) is involved in guiding receptor bearing cells to specific locations in the body, particularly to sites of inflammation, immune injury and immune dysfunction and is also associated with tissue damage, the induction of apoptosis, cell growth, and angiostasis. CXCR3 and its ligands are upregulated and highly expressed in diverse pathological situations including autoimmune disorders, inflammation, infection, transplant rejection, fibrosis, neurodegeneration and cancer.

A role of the CXCR3 axis in autoimmune disorders is corroborated by several preclinical and clinical observations. Autoimmune disorders in which histological analysis of inflammatory lesions or serum levels of patients revealed elevated levels of CXCR3 ligands or increased numbers of CXCR3 positive cells include rheumatoid arthritis (RA), systemic lupus erythematosus (SLE), lupus nephritis, multiple sclerosis (MS), inflammatory bowel disease (IBD; comprising Crohn's disease and ulcerative colitis), and type I diabetes mellitus (Groom, J. R. & Luster, A. D. Immunol Cell Biol 2011, 89, 207; Groom, J. R. & Luster, A. D. Exp Cell Res 2011, 317, 620; Lacotte, S., Brun, S., Muller, S. & Dumortier, H. Ann N Y Acad Sci 2009, 1173, 310). As expression of CXCR3 ligands is very low in healthy tissue, the above cited correlative evidence strongly suggests a role for CXCR3 in human autoimmune diseases. Further, Ruschpler and co-workers (Ruschpler, P. et al., Arthritis Res Ther 2003, 5(5), R241-252) describe elevated levels of the CXCR3 ligands in the synovial tissue of rheumatoid arthritis (RA) patients. Moreover, they show CXCR3 expression on mast cells and propose that these cells play a significant role in the pathology of RA. The authors state: “These findings suggest that substantial expression of CXCR3 protein on mast cells within synovial tissue from RA patients plays a significant role in the pathophysiology of RA, accompanied by elevated levels of the chemokines CXCL9 and CXCL10.” Mohan and co-workers (Mohan, K. et al., J Immunol 2007, 179(12), 8463-8469) propose that CXCR3 expression on T cells plays an essential role in recruitment of T cells to inflamed joints and contribute to the development of arthritis in an animal model for RA. Enghard and colleagues (Enghard, P. et al, Arthritis Rheum 2009, 60(1), 199-206) describe that CXCR3 expressing T cells are recruited into the inflamed kidneys and are also enriched in the urine of lupus patients. They propose that these CXCR3 expressing cells represent a valuable biomarker for nephritis activity in SLE and that the CXCR3 axis may represent a potential target for future therapy. Steinmetz and co-workers (Steinmetz, O. M. et al., J Immunol 2009, 183(7), 4693-4704) show that mice lacking the CXCR3 receptor develop an attenuated course of disease in a murine model for both SLE and lupus nephritis and propose CXCR3 as a promising therapeutic target in this disease. Further, Menke and colleagues (Menke, J. et al., J Am Soc Nephrol 2008, 19(6), 1177-1189) show that mice lacking CXCR3 or the CXCR3 ligand CXCL9 (MIG) develop a milder course of disease in two mouse models for lupus nephritis. Comini-Frota and colleagues (Comini-Frota, E. R. et al., CNS Drugs 2011, 25(11), 971-981) show the levels of the CXCR3 ligand CXCL10 correlate with pathological leasons (T1 and T2) in the CNS of multiple sclerosis (MS) patients. They conclude that their observation is in line with previous observations that CXCR3 expressing CD4 T cells in the peripheral blood of MS patients correlate with CNS lesions in MS patients. Further, a study by Uzawa and colleagues (Uzawa, A. et al., “Expression of chemokine receptors on peripheral blood lymphocytes in multiple sclerosis and neuromyelitis optica.” BMC Neurol 2010, 10, 113) shows that the percentage of CXCR3 expressing T cells in peripheral blood correlates with disease activity in MS patients. Moreover, Sporici and co-workers (Sporici, R. et al., Eur J Immunol 2010, 40(10), 2751-2761) present data from a pre-clinical model for MS that shows that CXCR3 expression on T cells is important for disease induction. Schroepf and colleagues (Schroepf, S. et al., Inflamm Bowel Dis 2010, 16(11), 1882-1890) present data that show that the CXCR3 axis (CXCR3 receptor and its ligands) is overexpressed in both pediatric Crohn's disease and pediatric ulcerative colitis. A study from Uno and co-workers (Uno, S. et al., Endocr J 2010, 57(11), 991-996) provides evidence that the CXCR3 axis plays a crucial role in the pathophysiology of Type I diabetes mellitus. The team could show that the CXCR3 ligand CXCL10 was expressed in the still intact beta cells in the pancreas of patients, and that infiltrating T cells express CXCR3. Hence, they conclude that the CXCL10 and CXCR3 interaction contributes to the pathology of Type I diabetes. Chen and co-workers (Chen, S. C. et al., Arch Dermatol Res 2010, 302(2), 113-123) could show that mRNA levels of CXCR3 and its ligands, CXCL9, -10 and -11, were significantly elevated in psoriatic lesions, as compared to non-lesional samples. The authors used quantitative image analysis to demonstrate a significant increase in the number of both epidermal and dermal CXCR3 expressing cells in lesional compared to non-lesional biopsies. The majority of CXCR3 expressing cells were located in the dermis were shown to be T lymphocytes. Taken together, the author concludes that ‘with its role in the trafficking of activated T lymphocytes into the dermis of psoriatic skin, CXCR3 represents a highly attractive target for the treatment of this disease’. Loos et al. (Loos, T. et al, Lab Invest 2006, 86(9), 902-916) describe that in synovial fluids of patients with spondylarthropathies (i.e. ankylosing spondylitis or psoriatic arthritis) or rheumatoid arthritis, significantly enhanced CXCL9 levels were observed. Thus, the authors conclude that the CXCR3 ligand CXCL9 is an important chemokine in autoimmune arthritis, including psoriatic arthritis. A study by Antonelli and co-workers (Antonelli, A. et al., Rheumatology (Oxford) 2008, 47(1), 45-49) demonstrates high serum levels of the CXCR3 binding chemokine CXCL10 in newly diagnosed systemic sclerosis. High values of CXCL10 were associated with a more severe clinical phenotype (lung and kidney involvement).

In the skin from vitiligo patients, pathogenic melanocyte-specific CD8 positive T cells were found to express CXCR3 (Boniface, K. et al. J Invest Dermatol 2018, 138(2), 355) and serum CXCL10 levels were associated with disease severity (Wang, X X. et al. Br J Dermatol 2016, 174(6), 1318). Studies in vitiligo mouse models emphasized a critical role of the CXCR3/CXCL10 axis in the pathogenesis of the disease. Blocking this axis by using anti-CXCL10 antibodies or using anti-CXCR3 depleting antibodies has been shown to prevent and reverse depigmentation in a vitiligo mouse model (Rashighi, M. et al. Sci Transl Med 2014, 6(223), 223ra23; Richmond J M et al. J Invest Dermatol 2017, 137(4), 982-5).

Preclinical disease models performed with CXCR3 deficient mice, mice deficient for one of the CXCR3 ligands or the use of antibodies blocking the function of either CXCR3 or one of its ligands further corroborate a role for the CXCR3 axis in immune pathology. For instance, it has been shown that mice deficient for either CXCR3 or the CXCR3 ligand CXCL9 show reduced pathology in a model for lupus nephritis (Menke, J. et al. J Am Soc Nephrol 2008, 19, 1177). Both clinical and pre-clinical evidence exists for the involvement of the CXCR3 axis in the pathology of interstitial cystitis. Sakthivel and co-workers (Sakthivel, S. K. et al., J Immune Based Ther Vaccines 2008, 6, 6.) could show that blocking the CXCR3 ligand CXCL10 in a mouse model for interstitial cystitis could reduce the severity of the disease. In the clinical setting, Ogawa and colleagues (Ogawa, T. et al., J Urol 2010, 183(3), 1206-1212) could show that CXCR3 binding chemokines are overrepresented in patient biopsies compared to healthy controls and might serve as biomarkers for interstitial cystitis. Similarly, blocking CXCL10 with an antibody reduced pathology in a rat model of rheumatoid arthritis (Mohan, K. & Issekutz, T. B. J Immunol 2007, 179, 8463). Similarly, in a murine model of inflammatory bowel disease, a blocking antibody against CXCL10 could prevent pathology in a therapeutic setting (Singh, U. P. et al. J Interferon Cytokine Res 2008, 28, 31). Further, experiments performed with tissue from CXCR3 deficient mice suggests a role for CXCR3 in celiac disease, another autoimmune type disorder (Lammers, K. M. et al. Gastroenterology 2008, 135, 194). Feferman and colleagues (Feferman, T. et al., J Neuroimmunol 2009, 209(1-2), 87-95) have previously demonstrated overexpression of CXCR3 and CXCL10 in myasthenia gravis and further investigated the role of CXCR3 and its ligand CXCL10 in a pre-clinical model of this disease. They found that by blocking either CXCL10 with a blocking antibody or CXCR3 with a small molecule antagonist they could suppress the pathology in this mouse model for myasthenia gravis. They conclude that blocking CXCR3/IP-10 signaling can be considered as a potential treatment modality for myasthenia gravis. Howard and colleagues (Howard, O. M. et al., Blood 2005, 105(11), 4207-4214) study tissue-specific self-antigens found in uveitis and show that these can be chemoattractants for normal human immunocytes, specifically, lymphocytes and immature DCs. They specifically show that these autoantigens induce CXCR3-expressing cells to migrate.

Inflammatory diseases that are associated with an elevated expression of the CXCR3 axis include chronic obstructive pulmonary disorder (COPD), asthma, sarcoidosis, atherosclerosis and myocarditis (Groom, J. R. & Luster, A. D. Immunol Cell Biol 2011, 89, 207; Groom, J. R. & Luster, A. D. Exp Cell Res 2011, 317, 620).

One study has shown that CXCR3 positive cells are increased in the lungs of smokers with COPD compared to healthy subjects and immunoreactivity for the CXCR3-ligand CXCL10 was present in the bronchiolar epithelium of smokers with COPD but not in the bronchiolar epithelium of smoking and nonsmoking control subjects (Saetta, M. et al. Am J Respir Crit Care Med 2002, 165, 1404). These findings suggest that the CXCR3 axis may be involved in the immune cell recruitment that occurs in peripheral airways of smokers with COPD. In agreement with these observations, a preclinical study of COPD revealed an attenuation of acute lung inflammation induced by cigarette smoke in CXCR3 deficient mice (Nie, L. et al. Respir Res 2008, 9, 82). There is pre-clinical evidence for the involvement of CXCR3 and CXCR3 binding chemokines in asthma. Suzaki and colleagues (Suzaki, Y. et al., Eur Respir J 2008, 31(4), 783-789) could show that a small molecule antagonist of the receptors CXCR3 and CCR5 could prevent the development of asthma features in a mouse model. Lin and co-workers (Lin, Y. et al., Respir Res 2011, 12, 123) performed a similar study in a CXCR3 deficient mice and concluded that CXCR3 may represent a novel therapeutic target for asthma. Busuttil and co-workers (Busuttil, A. et al., Eur Respir J 2009, 34(3), 676-686) studied the pathogenesis of pulmonary sarcoidosis and concluded that both lymphocytes and cells of monocyte linage express CXCR3 and are involved in the formation of sarcoid lung granulomas and that the CXCR3 ligands CXCL9 and CXCL11 are upregulated in the sarcoid bronchoalveolar fluid (BALF). Crescioli and co-workers (Crescioli, C. et al., Eur J Cell Biol 2012, 91(2), 139-149) analysed the pathophysiology of myocarditis and found that in the inflamed muscles, skeletal muscle cells actively secrete CXCL10, which in turn could attract CXCR3 expressing Th1 T cells and hence self-promote inflammation. They conclude that targeting CXCL10 might control the aberrant immune reaction in this condition. As CXCL10 is the cognate ligand for CXCR3, it can be reasoned that inhibiting CXCR3 with an antagonist would be beneficial. CXCR3 ligands are increased in plasma and lungs from patients developing acute respiratory distress syndrome. This increase was associated with severity of the disease (Jiang, Y. et al. Am J Respir Crit Care Med 2005, 171(8), 850; Bautista, E. et al. Exp Mol Pathol 2013, 94(3), 486; Yang, Y. et al. J Allergy Clin Immunol 2020, 146(1), 119-27 e4). The CXCR3 axis was shown to be a critical factor in the development of acute respiratory distress syndrome induced by different conditions such as acid aspiration, viral infection, sepsis, pneumonia in animal models. Knockdown of CXCL10 or CXCR3 or using anti-CXCL10 or anti-CXCR3 agents have been shown to ameliorate acute lung injury, reducing lung inflammation, lung edema and improving lung function (Ichikawa, A. et al. Am J Respir Crit Care Med 2013, 187(1), 65; Lang, S. et al. PLoS One 2017, 12(1), e0169100; Zhu, X. et al. J Surg Res 2016, 204(2), 288). In one investigation of atherosclerosis, CXCR3 expression was found on all T cells within human atherosclerotic lesions. CXCR3 ligands CXCL9, CXCL10 and CXCL11 were all found in endothelial and smooth muscle cells associated with those lesions, suggesting that they are involved in the recruitment and retention of CXCR3 positive cells, particularly activated T lymphocytes, observed within vascular wall lesions during atherogenesis (Mach, F. et al. J Clin Invest 1999, 104, 1041). Van Wanrooij and colleagues (van Wanrooij, E. J. et al., Arterioscler Thromb Vasc Biol 2008, 28(2), 251-257) could show that treatment with a CXCR3 antagonist results in attenuating atherosclerotic lesion formation in a mouse model by blocking direct migration of CXCR3 expressing effector immune cells from the circulation into the atherosclerotic plaque. Preclinical studies further support a role of CXCR3 in the development of atherosclerosis. CXCR3 genetic deletion in mice lacking ApoE results in a significantly reduced atherosclerotic lesion development within abdominal aortas (Veillard, N. R. et al. Circulation 2005, 112, 870).

Yoon and co-workers (Yoon, K. C. et al., Invest Ophthalmol Vis Sci 2010, 51(2), 643-650) investigated the components of the CXCR3 axis (CXCR3 and its ligands CXCL9,10,11) on the ocular surface of patients suffering from dry eye disease (also called dry eye syndrome). Both patients with inflammatory dry eye disease and patients with autoimmune dry eye disease (patients with Sjögren's syndrome) had elevated CXCR3 and CXCR3 ligands on the ocular surface.

Cameron and colleagues (Cameron, C. M. et al., J Virol 2008, 82(22), 11308-11317) tested the pandemic influenza strain H5N1 in ferrets, which is considered to be a relevant model to study influenza pathology. First, they describe that the CXCR3 ligand CXCL10 is highly upregulated following influenza infection. Next, they tested the effect of a small molecule CXCR3 antagonist on the course of H5N1 infection in ferrets. This resulted in a reduction of symptom severity and delayed mortality compared to vehicle treatment.

Campanella and co-workers (Campanella, G. S. et al., Proc Natl Acad Sci USA 2008, 105(12), 4814-4819) studied the influence of the CXCR3 axis on the development of cerebral malaria in a preclinical model. They conclude that CXCR3 on CD8 T cells is required for T cell recruitment into the brain and the development of murine cerebral malaria and suggest that the CXCR3 ligands CXCL9 and CXCL10 play distinct, nonredundant roles in the pathogenesis of this disease. Hence, it can be argued that a CXCR3 antagonist might represent a suitable therapeutic approach to treat cerebral malaria.

A pivotal role for the CXCR3 axis has also been suggested in rejection reactions after organ transplantation and bone marrow transplantation related toxicity (Groom, J. R. & Luster, A. D. Exp Cell Res 2011, 317, 620). Preclinically, CXCR3 deficient mice show a significant resistance to allograft rejection (Hancock, W. W. et al. J Exp Med 2000, 192, 1515). Romagnani and colleague (Romagnani, P. et al., Clin Chim Acta 2012, 413(17-18), 1364-1373) conclude in their literature review that the CXCR3 ligand CXCL10 is not only useful as a biomarker to predict the severity of rejection and to monitor the inflammatory status of organ recipients but also as a therapeutic target for graft-versus-host disease and transplanted organs in general. In a preclinical model for lung transplant rejection, Seung and colleagues (Seung, E. et al., J Immunol 2011, 186(12), 6830-6838) show that CXCR3 deficient effector T cells display a reduced capacity to induce fatal pulmonary inflammation and hence tissue rejection. The authors conclude that inhibition of CXCR3 on effector T cells may be therapeutically beneficial for the prevention of lung transplant rejection. Matl and colleagues (Matl, I. et al., Kidney Blood Press Res 2010, 33(1), 7-14) describe that patients with transplanted kidneys with high mRNA levels of the CXCR3 ligand CXCL10 (IP-10) were found to be at risk for premature kidney graft loss. It therefore is likely that CXCL10—and in extension its receptor CXCR3—is involved in the pathology of kidney transplant rejection and hence CXCR3 inhibition may be a therapeutic strategy for prevention of kidney graft loss. He and colleagues (He, S. et al., J Immunol 2008, 181(11), 7581-7592) show that blocking CXCR3 with an antibody shows beneficial effects in a pre-clinical model of graft-versus-host disease.

CXCR3 ligand plasma concentrations also positively correlate with diverse liver pathologies, including liver cirrhosis and fibrosis in humans (Tacke, F., et al. Liver Int 2011, 31(6), 840).

In the field of oncology, blocking the CXCR3 axis has been proposed to help limit the metastatic spread of cancer cells. For instance, administration of the small molecule CXCR3 receptor antagonist AMG487 could limit the metastasis of tumor cells to the lungs (Pradelli, E. et al. Int J Cancer 2009, 125, 2586). Walser and co-workers (Walser, T. C. et al., Cancer Res 2006, 66(15), 7701-7707) could show that a small molecular weight CXCR3 antagonist has the potential to inhibit tumor metastasis. In this particular study, the spread of a murine mammary tumor to the lung could be inhibited by CXCR3 inhibition. Functional evidence for a role of CXCR3 in regulating B-cell chronic lymphocytic leukemia (CLL) was reported by Trentin and coworkers (Trentin, L. et al. J Clin Invest 1999, 104, 115). Further, in a pre-clinical model for brain tumor/glioblastoma, Liu and co-workers (Liu, C. et al., Carcinogenesis 2011, 32(2), 129-137) showed that pharmacological inhibition of CXCR3 with a small molecule inhibitor increased the survival of tumor bearing mice. Previously, Maru and colleagues (Maru, S. V. et al., J Neuroimmunol 2008, 199(1-2), 35-45.) could show that brain tumor glioma cells showed increased production of CXCL10, as well as an increased expression of its receptor CXCR3. CXCL10 induced an ERK1/2-dependent increase in cell proliferation. Amy Fulton (Fulton, A. M. Curr Oncol Rep 2009, 11(2), 125-131) reviews the literature evidence about the involvement of CXCR3 in cancer. She states that CXCR3 has been detected on many malignant cell lines and has been linked to patient outcomes in colon and breast cancer as well as melanoma. In those conditions, high CXCR3 expression is linked to more aggressive disease.

In the central nervous system, blocking the CXCR3 axis may have beneficial effects and prevent neurodegeneration. Increased expression of CXCL10 in the CNS has been demonstrated in ischemia, Alzheimer's disease, multiple sclerosis (MS), and human immunodeficiency virus (HIV)-encephalitis. For example, two research groups describe a general role of CXCR3 and its ligand CXCL10 in neurodegenerative disorders and neuronal disfunction or neuronal death. Cho and colleagues (Cho, J. et al., J Neuroimmunol 2009, 207(1-2), 92-100) describe that rat neurons can express CXCR3 and that CXCL10 is able to alter neuronal functions. The authors postulate that this interaction between CXCR3 and CXCL10 may contribute to altered function of the central nervous system in chronic neuroinflammatory or neurodegenerative disorders. Van Weering and colleagues (van Weering, H. R. et al., Hippocampus 2011, 21(2): 220-232.) found that in mice region-specific role for CXCL10/CXCR3 signaling in neuron-glia and glia-glia interactions under pathological conditions. Using mice deficient for CXCR3, they conclude that CXCR3 on microglial cells is important for neuronal death under excitotoxic conditions. Taken together, these two publications show that CXCR3 and CXCL10 can lead to neuronal impairment or even death in pre-clinical models. Xia and colleagues (Xia, M. Q. et al., J Neuroimmunol 2000, 108(1-2), 227-235) show that the CXCR3 ligand CXCL10 was observed in a subpopulation of astrocytes in normal brain, and was markedly elevated in astrocytes in Alzheimer's disease brains. Moreover, they demonstrate that CXCL10 and CXCL9 can induce signals in rat neurons. Vergote and co-workers (Vergote, D. et al., Proc Natl Acad Sci USA 2006, 103(50), 19182-19187) observed that in patients suffering from HIV associated dementia, the chemokine CXCL12 exists in a truncated form missing the first four amino acids (CXCL12(5-67)). Unlike the full length CXCL12, this truncated form does no longer signal via the chemokine receptor CXCR4, but instead can now signal via CXCR3. The authors conclude that this novel interaction leads to neuronal pathology. In vivo, they could show that neuroinflammation, neuronal loss, and neurobehavioral abnormalities caused by the truncated form of CXCL12 were prevented by a CXCR3 antagonist. In a study looking to identify drug-like molecules that provide neuroprotection against HTT fragment-induced neurodegeneration in a model for Huntington's disease, two CXCR3 receptor antagonists were identified (Reinhart, P. H. et al. Neurobiol Dis 2011, 43, 248). Press and co-workers (Press, R. et al., J Clin Immunol 2003, 23(4), 259-267.) state that infiltration of spinal nerve roots and peripheral nerves by macrophages and T cells are rather consistent immunopathologic findings in patients with Guillain-Barre syndrome (GBS) and chronic inflammatory demyelinating polyneuropathy (CIDP). They investigated inflammatory mediators in the cerebrospinal fluid of GBS and CIDP patients and found that the levels of the CXCR3 ligand CXCL10 are elevated in both conditions and hypothesize that CXCL10 is implicated in the pathogenesis of these two disorders.

COMPOUND can be prepared according to the procedure given in WO 2016/113344.

It has now been found that certain crystalline forms of COMPOUND may under certain conditions be found. Said crystalline forms of COMPOUND are novel and may have advantageous properties in view of the potential use of COMPOUND as active pharmaceutical ingredient. Such advantages may include better flow properties; less hygroscopicity; better properties for tablet manufacturing (for example a sufficiently high melting point); better reproducibility in manufacturing (for example better filtration parameters, better reproducibility of formation, and/or better sedimentation); and/or defined morphology. Such crystalline forms of COMPOUND may be particularly suitable in a process of manufacturing certain pharmaceutical compositions. The crystalline form 1 of COMPOUND is a non-solvated, non-hydrated solid-state form and differs thus from the crystalline forms 2 and 3. As solvated solid-state forms tend to desolvate over time, a non-solvated form is advantageous as an uncontrolled desolvation is not possible. Another disadvantage of a solvated solid-state form is the amount of residual solvent content in a pharmaceutical composition that might even be above the recommended allowed daily intake (as specified by ICH recommendations).

### DESCRIPTION OF THE FIGURES

FIG. 1 shows the X-ray powder diffraction diagram of COMPOUND in the crystalline form 1, wherein the X-ray powder diffraction diagram was measured with the XRPD method 1 (as described in experimental procedures) and is displayed against Cu Kα radiation. In the diagram the angle of refraction 2θ is plotted on the horizontal axis and the counts on the vertical axis. The X-ray diffraction diagram shows peaks having a relative intensity, as compared to the most intense peak in the diagram, of the following percentages (relative peak intensities given in parenthesis) at the indicated angles of refraction 2theta (selected peaks from the range 3-30° 2theta with relative intensity larger than 10% are reported): 5.8° (20%), 8.9° (18%), 9.1° (13%), 11.3° (14%), 12.1° (37%), 12.7° (13%), 14.3° (100%), 15.5° (33%), 16.4° (15%), 16.7° (96%), 17.2° (64%), 17.9° (24%), 18.2° (25%), 18.5° (20%), 20.4° (13%), 20.7° (12%), 21.1° (21%), 21.3° (13%), 21.7° (11%), 22.3° (14%), 22.5° (20%), 23.3° (12%), 24.7° (28%), and 26.9° (25%).

FIG. 2 shows the X-ray powder diffraction diagram of COMPOUND in the crystalline form 2 as obtained from acetone, wherein the X-ray powder diffraction diagram was measured with the XRPD method 2 (as described in experimental procedures) and is displayed against Cu Kα radiation. In the diagram the angle of refraction 2θ is plotted on the horizontal axis and the counts on the vertical axis. The X-ray diffraction diagram shows peaks having a relative intensity, as compared to the most intense peak in the diagram, of the following percentages (relative peak intensities given in parenthesis) at the indicated angles of refraction 2theta (selected peaks from the range 3-30° 2theta with relative intensity larger than 10% are reported): 8.6° (11%), 9.6° (19%), 14.2° (22%), 14.7° (44%), 15.0° (22%), 16.0° (55%), 16.7° (100%), 17.3° (70%), 18.7° (26%), 20.5° (77%), 22.3° (53%), and 22.7° (33%).

FIG. 3 shows the X-ray powder diffraction diagram of COMPOUND in the crystalline form 3, wherein the X-ray powder diffraction diagram was measured with the XRPD method 2 (as described in experimental procedures) and is displayed against Cu Kα radiation. In the diagram the angle of refraction 2θ is plotted on the horizontal axis and the counts on the vertical axis. The X-ray diffraction diagram shows peaks having a relative intensity, as compared to the most intense peak in the diagram, of the following percentages (relative peak intensities given in parenthesis) at the indicated angles of refraction 2theta (selected peaks from the range 3-30° 2theta with relative intensity larger than 10% are reported): 8.7° (23%), 9.8° (25%), 11.4° (10%), 13.4° (16%), 14.2° (14%), 15.3° (13%), 16.4° (55%), 17.0° (100%), 17.7° (37%), 19.7° (13%), 20.6° (24%), 21.3° (40%), 21.8° (29%), and 28.5° (34%).

For the avoidance of any doubt, the above-listed peaks describe the experimental results of the X-ray powder diffraction shown in FIGS. 1 to 3. It is understood that, in contrast to the above peak list, only a selection of characteristic peaks is required to fully and unambiguously characterise the COMPOUND in the respective crystalline form of the present invention.

FIG. 4 shows the differential scanning calorimetry (DSC) thermogram of COMPOUND in the crystalline form 1. In the DSC thermogram of FIG. 4 the temperature (° C.) is plotted on the horizontal axis and the heat flow (mW) on the vertical axis.

FIG. 5 shows the gravimetric vapour sorption diagram of COMPOUND in the crystalline form 1 as obtained from Example 1.

In the gravimetric vapour sorption diagram of FIG. 5 the relative humidity (% RH) is plotted on the horizontal axis and the mass change (% dm) on the vertical axis.

FIG. 6 shows the thermogravimetric analysis (TGA) of COMPOUND in the crystalline form 2.

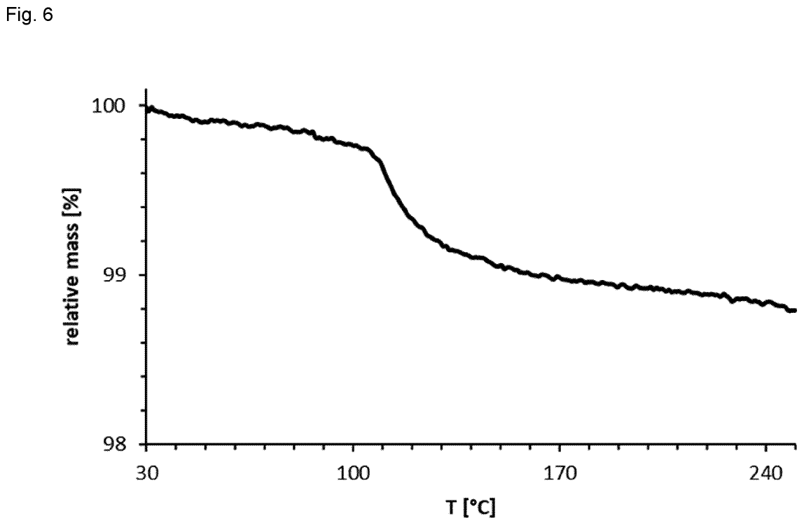

In the thermogravimetric analysis diagram of FIG. 6 the temperature (° C.) is plotted on the horizontal axis and the relative mass (%) on the vertical axis.

### DETAILED DESCRIPTION OF THE INVENTION

1) A first embodiment of the invention relates to a crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone (COMPOUND); characterized by the presence of peaks in the X-ray powder diffraction diagram at the following angles of refraction 2θ: 14.3°, 16.7°, and 17.2°.

It is understood that the crystalline form according to embodiment 1) comprise COMPOUND in a crystalline form of the free base (i.e. not in form of a salt).

Furthermore, the crystalline forms of COMPOUND may comprise non-coordinated and/or coordinated solvent (especially non-coordinated and/or coordinated water). Coordinated solvent (especially coordinated water) is used herein as term for a crystalline solvate (especially a crystalline hydrate). Likewise, non-coordinated solvent (especially water) is used herein as term for physiosorbed or physically entrapped solvent (especially water; definitions according to Polymorphism in the Pharmaceutical Industry (Ed. R. Hilfiker, V C H, 2006), Chapter 8: U. J. Griesser: The Importance of Solvates). Crystalline form 1 of COMPOUND comprises no coordinated water but may comprise non-coordinated water or another non-coordinated solvent.

COMPOUND in the crystalline form 1 has a melting point of T=169±3° C. as measured by DSC. COMPOUND in crystalline form 1 is not hygroscopic according to Ph. Eur. (European Pharmacopeia 10.0, section 5.11).

2) Another embodiment relates to a crystalline form of COMPOUND according to embodiment 1), characterized by the presence of peaks in the X-ray powder diffraction diagram at the following angles of refraction 2θ: 14.3°, 15.5°, 16.4°, 16.7°, and 17.2°.

3) Another embodiment relates to a crystalline form of COMPOUND according to embodiment 1), characterized by the presence of peaks in the X-ray powder diffraction diagram at the following angles of refraction 2θ: 5.8°, 8.9°, 12.1°, 14.3°, 15.5°, 16.4°, 16.7°, 17.2°, 18.5°, and 26.9°.

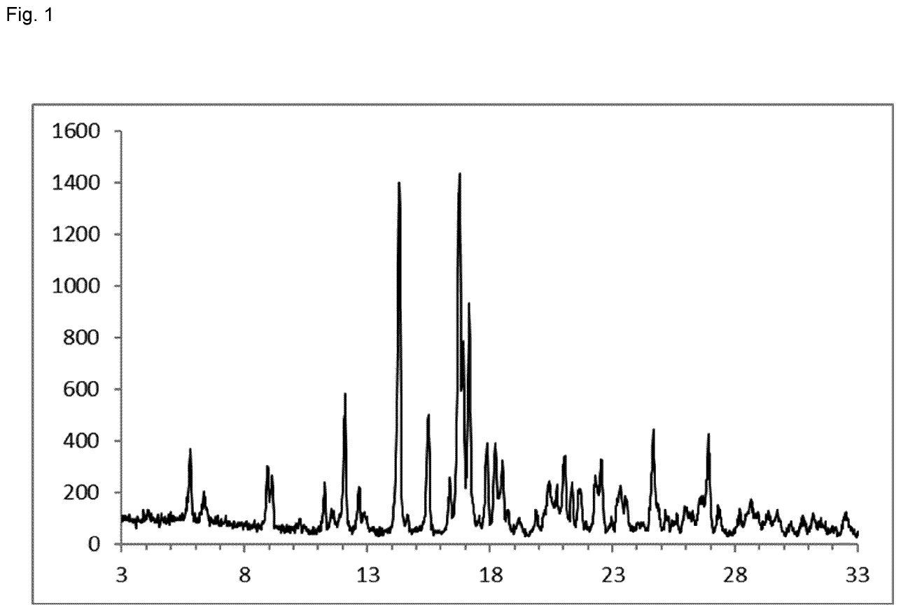

4) Another embodiment relates to a crystalline form of COMPOUND according to embodiment 1), which essentially shows the X-ray powder diffraction pattern as depicted in FIG. 1.

5) Another embodiment relates to a crystalline form of COMPOUND; characterized by the presence of peaks in the X-ray powder diffraction diagram at the following angles of refraction 2θ: 16.0°, 16.7°, and 20.5°.

Depending on the solvent used for the crystallization (acetone or THF), COMPOUND in crystalline form 2 may contain coordinated and/or non-coordinated solvent.

6) Another embodiment relates to a crystalline form of COMPOUND according to embodiment 5), characterized by the presence of peaks in the X-ray powder diffraction diagram at the following angles of refraction 2θ: 9.6°, 16.0°, 16.7°, 17.3°, and 20.5°.

7) Another embodiment relates to a crystalline form of COMPOUND according to embodiment 5), characterized by the presence of peaks in the X-ray powder diffraction diagram at the following angles of refraction 2θ: 8.6°, 9.6°, 14.7°, 15.0°, 16.0°, 16.7°, 17.3°, 18.7°, and 20.5°.

8) Another embodiment relates to a crystalline form of COMPOUND; characterized by the presence of peaks in the X-ray powder diffraction diagram at the following angles of refraction 2θ: 9.8°, 17.0°, and 17.7°.

COMPOUND in crystalline form 3 is an acetonitrile solvate.

9) Another embodiment relates to a crystalline form of COMPOUND according to embodiment 8), characterized by the presence of peaks in the X-ray powder diffraction diagram at the following angles of refraction 2θ: 8.7°, 9.8°, 13.4°, 17.0°, and 17.7°.

10) Another embodiment relates to a crystalline form of COMPOUND according to embodiment 8), characterized by the presence of peaks in the X-ray powder diffraction diagram at the following angles of refraction 2θ: 8.7°, 9.8°, 11.4°, 13.4°, 14.2°, 15.3°, 16.4°, 17.0°, 17.7°, and 19.7°.

11) Another embodiment relates to a crystalline form, such as an essentially pure crystalline form, of the compound 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone obtainable by mixing about 5 mg amorphous COMPOUND with about 0.02 mL solvent selected from ethyl acetate, iso-propanol or tert.-buty methyl ether and storing of the mixture for about 5 days.

12) Another embodiment relates to a crystalline form of COMPOUND according to embodiment 11); characterized by the presence of peaks in the X-ray powder diffraction diagram at the following angles of refraction 2θ: 14.3°, 16.7°, and 17.2°.

13) Another embodiment relates to a crystalline form of COMPOUND according to embodiment 11), characterized by the presence of peaks in the X-ray powder diffraction diagram at the following angles of refraction 2θ: 14.3°, 15.5°, 16.4°, 16.7°, and 17.2°.

14) Another embodiment relates to a crystalline form of COMPOUND according to embodiment 11), characterized by the presence of peaks in the X-ray powder diffraction diagram at the following angles of refraction 2θ: 5.8°, 8.9°, 12.1°, 14.3°, 15.5°, 16.4°, 16.7°, 17.2°, 18.5°, and 26.9°.

15) Another embodiment relates to a crystalline form of COMPOUND according to embodiment 11), which essentially shows the X-ray powder diffraction pattern as depicted in FIG. 1.

16) Another embodiment relates to a crystalline form of COMPOUND according to any one of embodiments 1) to 4) or 11) to 15), which has a melting point of about 169° C. (especially of 169±3° C.) as determined by differential scanning calorimetry.

The differential scanning calorimetry (DSC) data may be measured by heating a sample of COMPOUND (especially 1 to 5 mg) at 10° C. per minute in the range from −20° C. to 200° C. (and especially by the method as described in the experimental part).

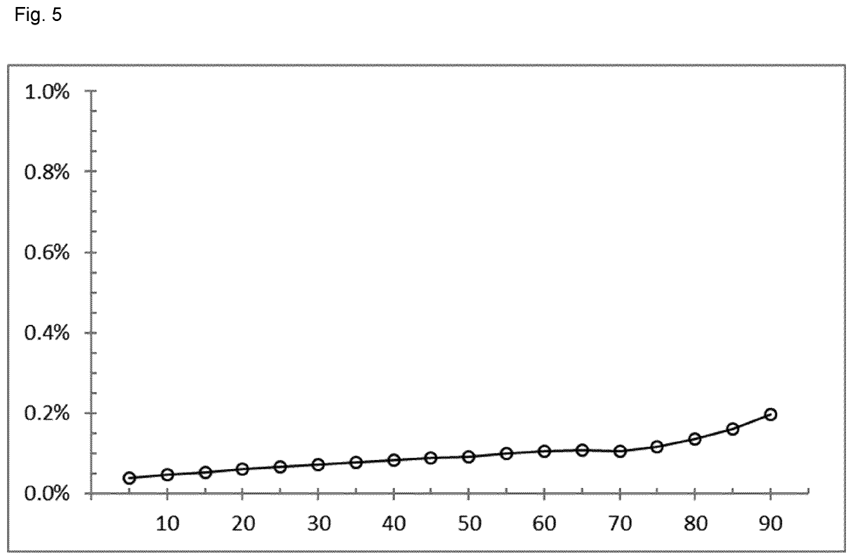

17) Another embodiment relates to a crystalline form of COMPOUND according to any one of embodiments 1) to 4) or 11) to 16), which essentially shows a gravimetric moisture sorption profile as depicted in FIG. 5, wherein the gravimetric moisture sorption profile is measured at about 25° C. (especially at 25° C.).

18) Another embodiment relates to the crystalline form of COMPOUND according to any one of embodiments 1) to 4), obtainable by the process of embodiment 11).

Based on the dependencies of the different embodiments 1) to 18) as disclosed hereinabove, the following embodiments are thus possible and intended and herewith specifically disclosed in individualised form:

1, 2+1, 3+1, 4+1, 5, 6+5, 7+5, 8, 9+8, 10+8, 11, 12+11, 13+11, 14+11, 15+11, 16+1, 16+2+1, 16+3+1, 16+4+1, 16+11, 16+12+11, 16+13+11, 16+14+11, 16+15+11, 17+1, 17+2+1, 17+3+1, 17+4+1, 17+11, 17+12+11, 17+13+11, 17+14+11, 17+15+11, 17+16+1, 17+16+2+1, 17+16+3+1, 17+16+4+1, 17+16+11, 17+16+12+11, 17+16+13+11, 17+16+14+11, 17+16+15+11, 18;

In the list above the numbers refer to the embodiments according to their numbering provided hereinabove whereas “+” indicates the dependency from another embodiment. The different individualised embodiments are separated by commas. In other words, “16+2+1” for example refers to embodiment 16) depending on embodiment 2), depending on embodiment 1), i.e. embodiment “16+2+1” corresponds to embodiment 1) further characterised by the features of the embodiments 2) and 16).

For avoidance of any doubt, whenever one of the above embodiments refers to “peaks in the X-ray powder diffraction diagram at the following angles of refraction 2θ”, said X-ray powder diffraction diagram is obtained by using combined Cu Kα1 and Kα2 radiation, without Kα2 stripping; and it should be understood that the accuracy of the 2θ values as provided herein is in the range of +/−0.1-0.2°. Notably, when specifying an angle of refraction 2theta (2θ) for a peak in the invention embodiments and the claims, the 2θ value given is to be understood as an interval from said value minus 0.2° to said value plus 0.2° (2θ+/−0.2°); and preferably from said value minus 0.1° to said value plus 0.1° (2θ+/−0.1°).

Where the plural form is used for compounds, solid, pharmaceutical compositions, diseases and the like, this is intended to mean also a single compound, solid, pharmaceutical composition, disease or the like.

Definitions provided herein are intended to apply uniformly to the subject matter as defined in any one of embodiments 1) to 18), and, mutatis mutandis, throughout the description and the claims unless an otherwise expressly set out definition provides a broader or narrower definition. It is well understood that a definition or preferred definition of a term or expression defines and may replace the respective term or expression independently of (and in combination with) any definition or preferred definition of any or all other terms or expressions as defined herein.

The term “enantiomerically enriched” is understood in the context of the present invention to mean especially that at least 90, preferably at least 95, and most preferably at least 99 percent by weight of the COMPOUND are present in form of one enantiomer of the COMPOUND. It is understood that COMPOUND is present in enantiomerically enriched absolute (R)-configuration.

The term “essentially pure” is understood in the context of the present invention to mean especially that at least 90, preferably at least 95, and most preferably at least 99 percent by weight of the crystals of COMPOUND are present in a crystalline form according to the present invention.

When defining the presence of peak in e.g. an X-ray powder diffraction diagram, a common approach is to do this in terms of the S/N ratio (S=signal, N=noise). According to this definition, when stating that a peak has to be present in an X-ray powder diffraction diagram, it is understood that the peak in the X-ray powder diffraction diagram is defined by having an S/N ratio (S=signal, N=noise) of greater than x (x being a numerical value greater than 1), usually greater than 2, especially greater than 3.

In the context with stating that the crystalline form essentially shows an X-ray powder diffraction pattern as depicted in FIG. 1, the term “essentially” means that at least the major peaks of the diagram depicted in said figure, i.e. those having a relative intensity of more than 20%, especially more than 10%, as compared to the most intense peak in the diagram, have to be present. However, the person skilled in the art of X-ray powder diffraction will recognize that relative intensities in X-ray powder diffraction diagrams may be subject to strong intensity variations due to preferred orientation effects.

Unless used regarding temperatures, the term “about” placed before a numerical value “X” refers in the current application to an interval extending from X minus 10% of X to X plus 10% of X, and preferably to an interval extending from X minus 5% of X to X plus 5% of X and especially to X. In the particular case of temperatures, the term “about” placed before a temperature “Y” refers in the current application to an interval extending from the temperature Y minus 5° C. to Y plus 5° C., preferably to an interval extending from Y minus 3° C. to Y plus 3° C., and especially to Y. Room temperature means a temperature of about 25° C.

Whenever the word “between” or “to” is used to describe a numerical range, it is to be understood that the end points of the indicated range are explicitly included in the range. For example: if a temperature range is described to be between 40° C. and 80° C. (or 40° C. to 80° C.), this means that the end points 40° C. and 80° C. are included in the range; or if a variable is defined as being an integer between 1 and 4 (or 1 to 4), this means that the variable is the integer 1, 2, 3, or 4.

The crystalline forms, especially the essentially pure crystalline forms, of COMPOUND according to any one of embodiments 1) to 18) can be used as medicaments, e.g. in the form of pharmaceutical compositions for enteral (such especially oral) or parenteral administration (including topical application or inhalation).

19) Another embodiment thus relates to a crystalline form of the compound 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone (COMPOUND) according to any one of embodiments 1) to 18) (especially 1) to 4) or 11) to 17)) for use as a medicament.

The crystalline solid, especially the essentially pure crystalline solid, of COMPOUND according to any one of embodiments 1) to 18) (especially 1) to 4) or 11) to 17)) may be used as single component or as mixture with other crystalline forms or amorphous form of COMPOUND.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the crystalline form of the present invention, optionally in combination with other therapeutically valuable substances, into a galenical administration form together with suitable, non-toxic, inert, pharmaceutically acceptable solid or liquid carrier materials and, if desired, usual pharmaceutical adjuvants.

20) A further embodiment of the invention relates to pharmaceutical compositions comprising as active ingredient a crystalline form of COMPOUND according to any one of embodiments 1) to 18) (especially a crystalline form of COMPOUND according to any one of embodiments 1) to 4) or 11) to 17)), and at least one pharmaceutically acceptable carrier material.

21) A further embodiment of the invention relates to a crystalline form of COMPOUND according to any one of embodiments 1) to 18) (especially according to any one of embodiments 1) to 4) or 11) to 17)), for use in the manufacture of a pharmaceutical composition, wherein said pharmaceutical composition comprises as active ingredient the COMPOUND, and at least one pharmaceutically acceptable carrier material.

The crystalline forms as defined in any one of embodiments 1) to 18) (especially the crystalline form as defined in any one of embodiments 1) to 4) or 11) to 17)) are useful for the prevention/prophylaxis or treatment of disorders relating to a dysfunction of the CXCR3 receptor or dysfunction of ligands signalling through CXCR3.

Such disorders relating to a dysfunction of the CXCR3 receptor or its ligands are diseases or disorders where a modulator of a human CXCR3 receptor is required. The above mentioned disorders may in particular be defined as comprising (auto-)immune/inflammatory mediated disorders; pulmonary disorders; cardiovascular disorders; infectious diseases; fibrotic disorders; neurodegenerative disorders; and tumor diseases.

22) A further embodiment of the invention relates to a crystalline form of COMPOUND according to any one of embodiments 1) to 18) (especially according to any one of embodiments 1) to 4) or 11) to 17)), for use in the prevention/prophylaxis or treatment of disorders selected from (auto-)immune/inflammatory mediated disorders; pulmonary disorders; cardiovascular disorders; infectious diseases; fibrotic disorders; neurodegenerative disorders; or tumor diseases.

(Auto-)immune/inflammatory mediated disorders may be defined as comprising rheumatoid arthritis (RA); multiple sclerosis (MS); inflammatory bowel disease (IBD; comprising Crohn's disease and ulcerative colitis); primary biliary cirrhosis (PBC); autoimmune hepatitis; systemic lupus erythematosus (SLE); lupus nephritis; antiphospholipid syndrome; Sjögren Syndrome; sarcoidosis; systemic sclerosis; spondylarthritis; psoriasis; psoriatic arthritis; interstitial cystitis; celiac disease; thyroiditis such as Hashimoto's thyroiditis, lymphocytic thyroiditis, Grave's disease; myasthenia gravis; type I diabetes; uveitis; episcleritis; scleritis; Kawasaki's disease; uveo-retinitis; posterior uveitis; uveitis associated with Behcet's disease; uveomeningitis syndrome; vitiligo; allergic encephalomyelitis; atopic diseases such as rhinitis, conjunctivitis, dermatitis; post-infectious autoimmune diseases including rheumatic fever and post-infectious glomerulonephritis; myopathies (comprising inflammatory myopathies); obesity and transplant related disorders. Transplant related disorders may be defined as comprising transplant rejection such as rejection of transplanted organs such as kidney, liver, heart, lung, pancreas, cornea, and skin; acute and/or chronic graft-versus-host diseases; and chronic allograft vasculopathy.

Pulmonary disorders may be defined as comprising acute lung injury; acute respiratory distress syndrome; asthma; and chronic obstructive pulmonary disorder (COPD).

Cardiovascular disorders may be defined as comprising atherosclerosis; and myocarditis.

Infectious diseases may be defined as comprising diseases mediated by various infectious agents and complications resulting threrefrom; such as malaria, cerebral malaria, leprosy, tuberculosis, influenza, Toxoplasma gondii, dengue, hepatitis B and C, herpes simplex, leishmania, Chlamydia trachomatis, lyme disease, and west nile virus.

Fibrotic disorders may be defined as comprising liver cirrhosis, idiopathic pulmonary fibrosis, renal fibrosis, endomyocardial fibrosis, systemic sclerosis, and arthrofibrosis.

Neurodegenerative disorders may be defined as comprising neurodegeneration and conditions involving neuronal death such as multiple sclerosis (including relapsing remitting multiple sclerosis and progressive multiple sclerosis), Alzheimer's disease, Parkinson's disease, Huntington's chorea, HIV associated dementia, prion mediated neurodegeneration, epilepsy, stroke, cerebral ischemia, cerebral palsy, neuromyelitis optica, clinically isolated syndrome, Alpers' disease, amyotrophic lateral sclerosis (ALS), senile dementia, dementia with Lewy bodies, Rett syndrome, spinal cord trauma, traumatic brain injury, trigeminal neuralgia, chronic inflammatory demyelinating polyneuropathy, Guillain-Barre syndrome, narcolepsy, glossopharyngeal neuralgia, mild cognitive decline, cognitive decline, spinal muscular atrophy, and cerebral malaria.

Tumor diseases may be defined as comprising all sorts of cancers such as large intestine cancer, rectal cancer, breast cancer, lung cancer, non-small cell lung cancer, prostate cancer, esophagal cancer, stomach cancer, liver cancer, bile duct cancer, spleen cancer, kidney cancer, urinary bladder cancer, uterine cancer, ovarian cancer, cervical cancer, testicular cancer, thyroid cancer, pancreas cancer, brain tumor, blood tumor, basophil adenoma, prolactinoma, hyperprolactinemia, adenomas, endometrial cancer, colon cancer;

chronic lymphocytic leukemia (CLL); and (especially) the metastatic spread of cancer.

23) A preferred embodiment of the invention relates to a crystalline form of COMPOUND according to any one of embodiments 1) to 18) (especially according to any one of embodiments 1) to 4) or 11) to 17)), for use in the prevention/prophylaxis or treatment of disorders selected from one, several or all of the following groups of diseases and disorders:
1) (Auto-)immune/inflammatory mediated diseases selected from rheumatoid arthritis, multiple sclerosis, Crohn's disease, ulcerative colitis, primary biliary cirrhosis, autoimmune hepatitis, systemic lupus erythematosus, lupus nephritis, Sjögren Syndrome, sarcoidosis, systemic sclerosis, spondylarthritis, psoriasis, psoriatic arthritis, interstitial cystitis, celiac disease, myasthenia gravis, type I diabetes, uveitis, inflammatory myopathies, dry eye disease, thyroiditis including Grave's disease, vitiligo, transplant rejection, acute and/or chronic graft versus host disease, and (skin) fibrosis;
2) Pulmonary diseases selected from acute lung injury, acute respiratory distress syndrome, asthma, and chronic obstructive pulmonary disorder;
3) Cardiovascular diseases selected from atherosclerosis, and myocarditis;
4) Infectious diseases selected from influenza, and cerebral malaria;
5) Fibrotic disorders selected from liver cirrhosis;
6) Neurodegenerative disorders selected from Alzheimer's disease, neurodegeneration, Huntington's chorea, neuromyelitis optica, chronic inflammatory demyelinating polyneuropathy, and Guillain-Barré syndrome;
7) Tumor diseases selected from brain tumor, colon cancer, breast cancer, and metastatic spread of cancer.

24) Another preferred embodiment of the invention relates to a crystalline form of COMPOUND according to any one of embodiments 1) to 18) (especially according to any one of embodiments 1) to 4) or 11) to 17)), for use in the prevention/prophylaxis or treatment of disorders selected from rheumatoid arthritis, multiple sclerosis, Crohn's disease, ulcerative colitis, systemic lupus erythematosus, lupus nephritis, sarcoidosis, systemic sclerosis, psoriasis, psoriatic arthritis, interstitial cystitis, celiac disease, myasthenia gravis, type I diabetes, vitiligo, uveitis, inflammatory myopathies, dry eye disease, thyroiditis including Grave's disease, transplant rejection, acute and/or chronic graft versus host disease, acute lung injury, acute respiratory distress syndrome, asthma, chronic obstructive pulmonary disorder, atherosclerosis, myocarditis, influenza, cerebral malaria, liver cirrhosis, Alzheimer's disease, neurodegeneration, Huntington's chorea, neuromyelitis optica, chronic inflammatory demyelinating polyneuropathy, Guillain-Barre syndrome, brain tumor, colon cancer, breast cancer, and metastatic spread of cancer.

For avoidance of any doubt, if a crystalline form of COMPOUND is described as useful for the prevention/prophylaxis or treatment of certain diseases, such a crystalline form of COMPOUND is likewise suitable for use in the preparation of a medicament for the prevention/prophylaxis or treatment of said diseases. Especially, if a crystalline form of COMPOUND is described as useful for the prevention/prophylaxis or treatment of diseases according to any one of embodiments 22) to 24), such a crystalline form of COMPOUND is likewise suitable for use in the preparation of a medicament for the prevention/prophylaxis or treatment of said diseases.

The present invention also relates to a method for the prevention/prophylaxis or treatment of a disease mentioned herein, comprising administering to a subject in need thereof (especially a patient in need thereof) a pharmaceutically active amount of a crystalline form of COMPOUND according to any one of embodiments 1) to 18) (especially according to any one of embodiments 1) to 4) or 11) to 17)), or of a pharmaceutical composition according to embodiment 20).

The present invention also relates to a method for manufacturing a pharmaceutical composition comprising as active ingredient COMPOUND and at least one pharmaceutically acceptable carrier material, wherein the manufacturing of the pharmaceutical composition comprises the step of admixing a crystalline form of COMPOUND according to any one of embodiments 1) to 18) (especially according to any one of embodiments 1) to 4) or 11) to 17)) with the at least one pharmaceutically acceptable carrier material.

The present invention also relates to a process for the preparation of COMPOUND in enantiomerically enriched form, and to processes for the preparation and characterization of the crystalline forms of COMPOUND according to any one of embodiments 1) to 18) (especially according to any one of embodiments 1) to 4) or 11) to 17)). Said processes are described in embodiment 11), as well as in the procedures of the experimental part below.

### Experimental Procedures

### Abbreviations (as Used Hereinbefore or Hereinafter)

CNS Central Nervous System
DSC Differential scanning calorimetry
EtOAc Ethyl acetate
FIG. Figure
GVS Gravimetric vapor sorption
HPLC High performance liquid chromatography
MeCN Acetonitrile
MeOH Methanol
min Minute(s)
NMR Nuclear Magnetic Resonance
RH relative humidity
s Second(s)
tBME tert.-butyl methyl ether
TGA thermogravimetric analysis
THF Tetrahydrofuran
XRPD X-ray powder diffraction

All solvents and reagents are used as obtained from commercial sources unless otherwise indicated.

Temperatures are indicated in degrees Celsius (° C.). Unless otherwise indicated, the reactions take place at room temperature (RT).

In mixtures, relations of parts of solvent or eluent or reagent mixtures in liquid form are given as volume relations (v/v), unless indicated otherwise.

X-Ray Powder Diffraction Analysis (XRPD)

XRPD Method 1

X-ray powder diffraction patterns were collected on a Bruker D8 Advance X-ray diffractometer equipped with a Lynxeye detector operated in reflection mode (coupled two Theta/Theta). Typically, the Cu X-ray tube was run at of 40 kV/40 mA. A step size of 0.02° (2θ) and a step time of 76.8 sec over a scanning range of 3-50° in 2θ were applied. The divergence and the antiscatter slit were set to fixed 0.3°. Powders were slightly pressed into a silicon single crystal sample holder with depth of 0.5 mm and samples were rotated in their own plane during the measurement. Diffraction data are reported using Cu Kα (λ=1.5418 Å) radiation. The accuracy of the 2θ values as provided herein is in the range of +/−0.1-0.2° as it is generally the case for conventionally recorded X-ray powder diffraction patterns.

XRPD Method 2

X-ray powder diffraction patterns were collected on a Bruker D8 GADDS-HTS diffractometer equipped with an automated XYZ stage, laser video microscope for auto-sample positioning and a Vantec-500 detector operated in reflection mode. Typically, the Cu X-ray tube is run at 40 kV/40 mA. X-ray optics consists of a single Göbel multilayer mirror coupled with a pinhole collimator of 0.5 mm. Typically a single frame was recorded over 180 s with goniometer positions of theta1 at 4° and theta2 at 16° and detector distance of 20 cm. The frame was integrated in the range of 5-35° 2θ. Samples run under ambient conditions were prepared as flat plate specimens using powder as received without grinding. Approximately 5-10 mg of sample was lightly pressed on a glass slide to obtain a flat surface. The sample was not moved over the measurement time. Diffraction data are reported using Cu Kα (λ=1.5418 Å) radiation. The accuracy of the 2θ values as provided herein is in the range of +/−0.1-0.2° as it is generally the case for conventionally recorded X-ray powder diffraction patterns.

Differential Scanning Calorimetry (DSC)

DSC data were collected on a PerkinElmer DSC8500 with Pyris Software 2.1.1.0106. The instrument was calibrated for energy and temperature using certified indium. Typically, 1-5 mg of a sample was heated at 10° C. min-1 in the range from −20° C. to 200° C. in a non-hermetic aluminum pan. A nitrogen purge of 20 mL min-1 was maintained over the sample. Peak temperatures are reported for melting points.

Gravimetric Vapor Sorption (GVS)

Moisture sorption isotherm was collected on a dynamic vapor sorption analyzer IGASORP HAS-036-080 from Hiden Isochema operated with Isochema HIsorp 2019 software version 4.02.0070. Typically, about 30 mg of a sample is placed in the sample holder and is submitted to a stepwise equilibration at defined relative humidity (RH) setpoints at 25° C. The sample mass is recorded at these setpoints and are used to build the moisture sorption isotherm. The relative humidity setpoints used were 40% to 0% RH followed by 0% to 95% RH in 5% RH intervals. Data shown consist of the moisture sorption isotherm in the range 5% to 90% of the sample submitted to the increasing relative humidity. The variation in mass between 40% relative humidity and 80% relative humidity in the first sorption scan is evaluated for the hygroscopicity determination. The classification is done in analogy to the European Pharmacopeia (Ph. Eur.) 10.0, section 5.11.

Thermogravimetric Analysis (TGA)

TGA data were collected on a Mettler Toledo STARe System (TGA/SDTA851e module). Typically, about 5 mg of a sample was heated at 10° C./min in the range from 30° C. to 250° C. in an automatically pierced standard TGA aluminum pan. A nitrogen gas purge was maintained over the sample during measurement.

I-Chemistry

COMPOUND can be prepared according to the procedure given in WO 2016/113344 (example 35).

### Reference Example 1

0.2 mL of a solution of 25 mg/mL of COMPOUND in MeOH is dispensed into 4 mL glass vials and is evaporated in a Combindancer device (Hettich, Switzerland) to yield 5 mg of amorphous transparent films of COMPOUND per vial.

### Example 1: COMPOUND in Crystalline Form 1

In a standard 4 mL glass vial 0.02 mL of EtOAc, iso-propanol or tBME is added to 5 mg of amorphous COMPOUND as obtained by Reference Example 1. After closing and vortexing for about 30 seconds the vial is stored for 5 days in the dark. The obtained solid when analyzed without drying is COMPOUND in crystalline form 1.

In another experiment 1 mL of a MeCN/tBME 1:2 v/v mixture is added to 100 mg of COMPOUND and the mixture is heated to 55° C. with a heating rate of 0.1° C./min and cooled to 20° C. with a cooling rate of 0.1° C./min. After overnight standing, the solid is isolated by filter centrifugation and dried for 15 min at 40° C./10 mbar to give COMPOUND in crystalline form 1.

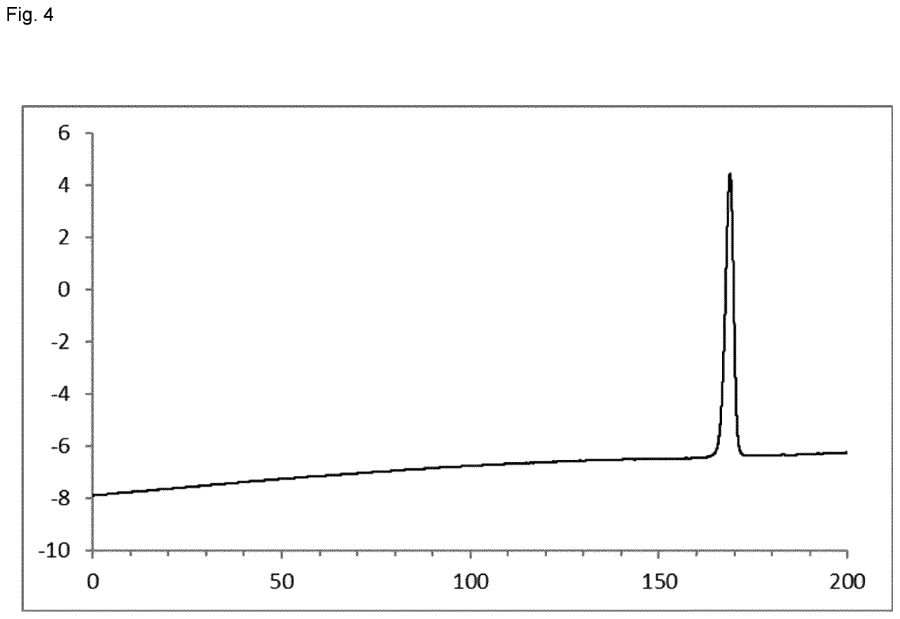

TABLE 1
Characterisation data for COMPOUND in crystalline form 1

| Technique | Data Summary | Remarks |
|---|---|---|
| XRPD | Crystalline | see FIG. 1 |
| 1H-NMR | Consistent | |
| DSC | Melting point: T = 169 ± 3° C. | see FIG. 4 |
| GVS | Not hygroscopic | See FIG. 5 |
| (hygroscopicity) | | |

### Example 2: COMPOUND in Crystalline Form 2

0.15 mL of acetone is added to 150 mg of COMPOUND (e.g., as obtained from Example 1) in a standard glass HPLC vial, and the suspension in the closed vial is shaken at 25° C. for 24 hours. The obtained solid is COMPOUND in crystalline form 2.

Thermogravimetric analysis of the solid residue after isolation by filter-centrifugation and 1 hour drying at 10 mbar shows an about 1.1% stepwise weight loss of acetone, which confirms the solvated nature of crystalline form 2.

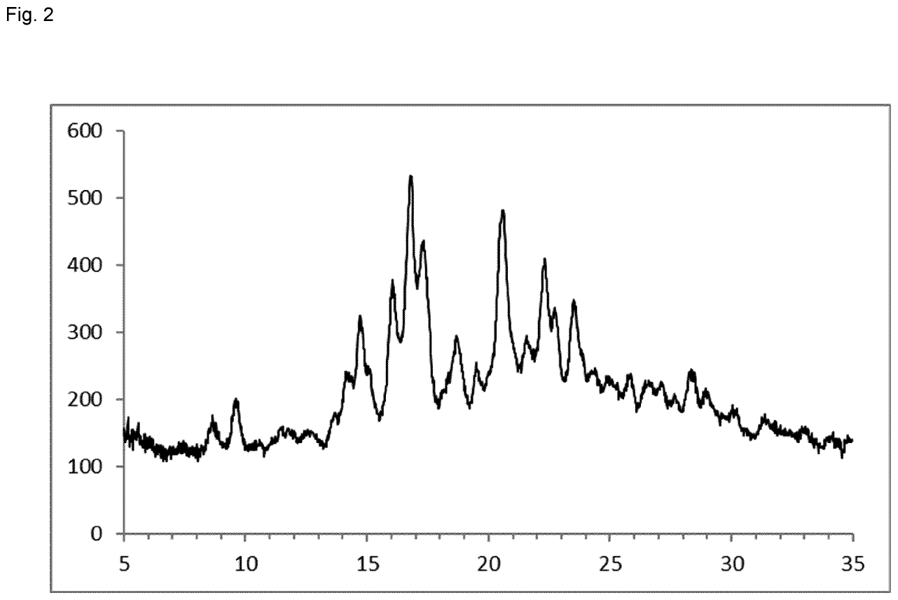

TABLE 2
Characterisation data for COMPOUND in crystalline form 2

| Technique | Data Summary | Remarks |
|---|---|---|
| XRPD | Crystalline | see FIG. 2 |
| TGA | Solvated form | see FIG. 6 |

### Example 3: COMPOUND in Crystalline Form 3

In a standard 4 mL glass vial 0.02 mL of MeCN is added to 5 mg of amorphous COMPOUND as obtained by Reference Example 1. After closing and vortexing for about 30 seconds the vial is stored for 5 days in the dark. The obtained solid when analyzed without drying is COMPOUND in crystalline form 3.

In another experiment 0.04 mL of MeCN is added to 20 mg of COMPOUND (e.g. as obtained from Example 1) in a standard glass HPLC vial, and the suspension in the closed vial is submitted to a temperature variation cycle while being stirred with a magnetic stirring bar. Temperature cycling (repetition of heating 20° C. to 40° C. in 1 hour and cooling 40° C. to 20° C. in 4 hours) and stirring for 25 hours is done in a Polar Bear device (Cambridge reactor design, UK) The obtained solid, when analyzed without drying, is COMPOUND in crystalline form 3.

Form 3 is observed by XRPD when measured still in presence of sufficient MeCN. Isolation by filter centrifugation or isolation and drying under reduced pressure leads to the transformation of crystalline form 3 to crystalline form 1, pointing to the metastable nature of form 3 in absence of sufficient MeCN. The combination of the facts that crystalline form 3 is only observed in presence of sufficient MeCN, that crystalline form 3 is obtained from crystalline form 1 in a suspension state, and that crystalline form 3 is metastable in absence of sufficient MeCN indicates that crystalline form 3 is a MeCN solvated structure.

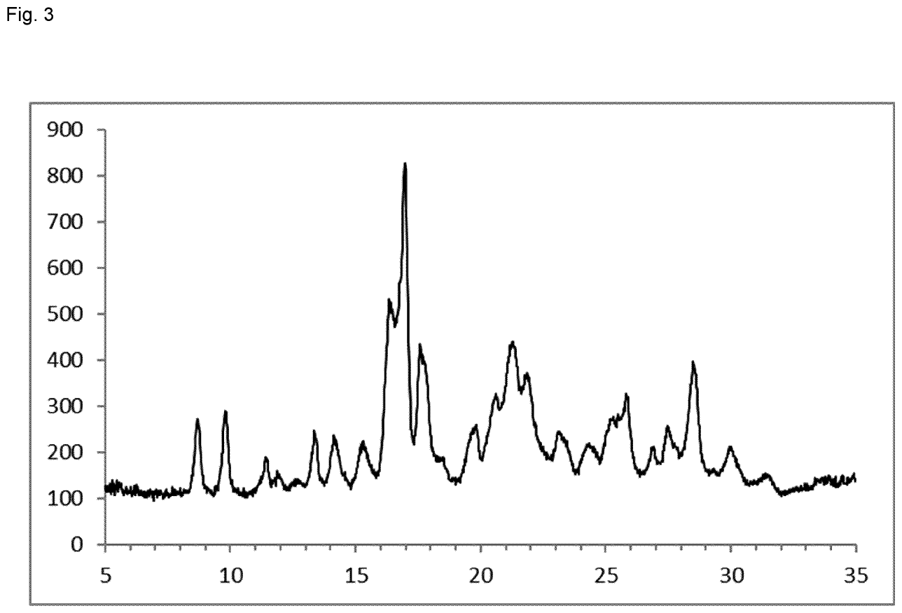

TABLE 3
Characterisation data for COMPOUND in crystalline form 3

| Technique | Data Summary | Remarks |
|---|---|---|
| XRPD | Crystalline | see FIG. 3 |

1. A crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, characterized by the presence of peaks in the X-ray powder diffraction diagram at the following angles of refraction 2θ: 14.3°, 16.7°, and 17.2°.

2. A crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone according to claim 1, characterized by the presence of peaks in the X-ray powder diffraction diagram at the following angles of refraction 2θ: 14.3°, 15.5°, 16.4°, 16.7°, and 17.2°.

3. A crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone according to claim 1, characterized by the presence of peaks in the X-ray powder diffraction diagram at the following angles of refraction 2θ: 5.8°, 8.9°, 12.1°, 14.3°, 15.5°, 16.4°, 16.7°, 17.2°, 18.5°, and 26.9°.

4. A crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone according to claim 1, which essentially shows the X-ray powder diffraction pattern as depicted in FIG. 1.

5-8. (canceled)

9. A crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone according to claim 1, wherein the crystalline form has a melting point of about 169° C. as determined by differential scanning calorimetry.

10-11. (canceled)

12. A pharmaceutical composition, wherein the pharmaceutical composition comprises as active ingredient a crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone and at least one pharmaceutically acceptable carrier material, wherein the crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone is characterized by the presence of peaks in the X-ray powder diffraction diagram at the following angles of refraction 2θ: 14.3°, 16.7°, and 17.2°.

13. A method for manufacturing a pharmaceutical composition comprising 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone and at least one pharmaceutically acceptable carrier material, wherein the method comprises admixing a crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone with the at least one pharmaceutically acceptable carrier material; and wherein the crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone is characterized by the presence of peaks in the X-ray powder diffraction diagram at the following angles of refraction 2θ: 14.3°, 16.7°, and 17.2°.

14. A method for the prevention or treatment of disorders selected from (auto-)immune/inflammatory mediated disorders; pulmonary disorders; cardiovascular disorders; infectious diseases; fibrotic disorders; neurodegenerative disorders; and tumor diseases, wherein the method comprises administering a crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone, wherein the crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone is characterized by the presence of peaks in the X-ray powder diffraction diagram at the following angles of refraction 2θ: 14.3°, 16.7°, and 17.2°.

15. (canceled)

16. A crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone according to claim 2, wherein the crystalline form has a melting point of about 169° C. as determined by differential scanning calorimetry.

17. A crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone according to claim 3, wherein the crystalline form has a melting point of about 169° C. as determined by differential scanning calorimetry.

18. A pharmaceutical composition according to claim 12, wherein the crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone is characterized by the presence of peaks in the X-ray powder diffraction diagram at the following angles of refraction 2θ: 5.8°, 8.9°, 12.1°, 14.3°, 15.5°, 16.4°, 16.7°, 17.2°, 18.5°, and 26.9°.

19. A pharmaceutical composition according to claim 12, wherein the crystalline form has a melting point of about 169° C. as determined by differential scanning calorimetry.

20. A method according to claim 13, wherein the crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone is characterized by the presence of peaks in the X-ray powder diffraction diagram at the following angles of refraction 2θ: 5.8°, 8.9°, 12.1°, 14.3°, 15.5°, 16.4°, 16.7°, 17.2°, 18.5°, and 26.9°.

21. A method according to claim 13, wherein the crystalline form has a melting point of about 169° C. as determined by differential scanning calorimetry.

22. A method according to claim 14, wherein the crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone is characterized by the presence of peaks in the X-ray powder diffraction diagram at the following angles of refraction 2θ: 5.8°, 8.9°, 12.1°, 14.3°, 15.5°, 16.4°, 16.7°, 17.2°, 18.5°, and 26.9°.

23. A method according to claim 14, wherein the crystalline form has a melting point of about 169° C. as determined by differential scanning calorimetry.

24. A method according to claim 14, wherein the disorder is type I diabetes.

25. A method according to claim 24, wherein the crystalline form of 1-{(R)-2-(2-Hydroxy-ethyl)-4-[2-trifluoromethyl-4-(2-trifluoromethyl-pyrimidin-5-yl)-thiazol-5-yl]-piperazin-1-yl}-2-(3-methyl-[1,2,4]triazol-1-yl)-ethanone is characterized by the presence of peaks in the X-ray powder diffraction diagram at the following angles of refraction 2θ: 5.8°, 8.9°, 12.1°, 14.3°, 15.5°, 16.4°, 16.7°, 17.2°, 18.5°, and 26.9°.

26. A method according to claim 24, wherein the crystalline form has a melting point of about 169° C. as determined by differential scanning calorimetry.
